(19) United States
(12) Patent Application Publication
(10) Pub. No.: US 2010/0292584 A1
(43) Pub. Date: Nov. 18, 2010

(54) SPECTRUM ANALYTICAL METHOD FOR QUANTIFYING HEAT-LUNG INTERACTION

(76) Inventor: Chih-Hsin Lee, Banciao City (TW)

Correspondence Address: APEX JURIS, PLLC, 12733 LAKE CITY WAY NORTHEAST, SEATTLE, WA, 98125, US

(21) Appl. No.: 12/467,108

(22) Filed: May 15, 2009

(51) Int. Cl. A61B 5/02

(52) U.S. Cl. 600/485; 600/504

(57) ABSTRACT

The present invention is related to a spectrum analytical method for quantifying hear-lung interaction, which can estimate cardiac function by using a heart-associated monitoring signal. According to the method of the present invention, quantification of heart-lung interaction is conducted by choosing spectrum signals within a specified frequency band, such that the interference to the heart-associated monitoring signals by incidental events occurring at a low frequency, can be avoided. Therefore, the method of the present invention can be performed even in the subjects who are not in a state of general anesthesia or sedation, and hence is very useful in estimating the cardiac function of the test subjects.

### FIELD OF INVENTION

The present invention is related to a spectrum analytical method for quantifying heart-lung interaction; more particularly, a method for quantifying heart-lung interaction by spectrum analysis of heart-associated monitoring signals.

### BACKGROUND OF INVENTION

Estimation of cardiac function is mainly based on hemodynamic parameters. Hemodynamic parameters are also life signs. For example, hypovolemic shock is usually initiated by decrease in blood volume (BV), dramatic decrease in cardiac output (CO) and increase in peripheral vascular resistance.

Cardiac preload, i.e. cardiac blood volume, means cardiac load before myocardial contraction, corresponding to ventricular end-diastolic volume or ventricular end-diastolic wall tension. Preload is an important factor for modulating stroke volume (SV), and stroke volume is one of the determinants for cardiac output. Cardiac output per minute is a product of stroke volume per beat and heart rate (HR, i.e. beats/minute), namely, CO=SV×HR.

As preload increases, namely the initial length of myocardial fiber increases, myocardial contractility elevates and hence stroke volume per beat increases. However, if preload excessively increases and the initial length of myocardial fiber is beyond its optimal range, myocardial contractility may reduce and stroke volume per beat may decrease. If heart is expanded, leading to increase in cardiac blood volume and the length of myocardial fiber, myocardial contractility during the next heart beat will increase according to Starling's Law, resulting in increased left ventricular stroke volume and hence, increased cardiac output. Therefore, cardiac blood volume (i.e. preload) is an important predictor for cardiac function.

Conventionally used estimations of cardiac blood volume have some limitations in their use. In one method for estimating cardiac blood volume, central venous pressure is used as the predictor. The pressure at the orifice of right atrium is measured according to U-tube principle by a pressure meter provided at the end of a central vena catheter. However, left ventricular pressure is most concerned clinically. It is not accurate enough for clinical use to predict left ventricular pressure from right atrial pressure, especially in the patients with acute or chronic cardiac/pulmonary co-morbidities. If the pressure and the volume of left ventricle are measured directly, said measurement should be conducted through cardiac catheterization in a cardiac catheterization room. Cardiac catheterization is costly and both the operators and the patient need to be exposed to high level of radiation. In addition, catheterization procedures are complicated and time-consuming and hence may not be applicable in critically ill patients who need emergent treatment.

Swan ganz pulmonary artery catheters have also been used in indirect measurement of left atrial pressure, from which the blood volume of left ventricle can be estimated. The Swan ganz catheter is drifted from a peripheral vein through right heart into one branch of a pulmonary artery, then the balloon is wedged into the pulmonary artery. The measured pressure is called “pulmonary artery wedge pressure, PAWP”, from which left atrial pressure can be indirectly estimated; however, accuracy may decrease in patients with pulmonary arterial diseases or pulmonary diseases. In the course of measurement, the catheter needs to pass through two cardiac valves and enter into the branch of the pulmonary artery. Such procedures are difficult to be performed without the aid of a fluoroscope; however, there are few wards or intensive care units provided with such radiographic apparatuses. Furthermore, even if the fluoroscope is available, the operators and the patient may be exposed to high level of radiation. Therefore, it is difficult for the Swan ganz pulmonary artery catheter to be used in subjects with emergent or severe diseases. In addition, some clinical researches have revealed that among the patients subjected to monitoring by the pulmonary artery wedge pressure, mortality was not reduced whereas the patients with multiple complications increased. In view of the above, new therapeutic guidelines no more recommends using pulmonary artery catheters. In another aspect, higher pressure does not always represent higher cardiac blood volume; a failing heart with compromised myocardial compliance may also lead to increased pressure. Furthermore, the measurements obtained by this method are easily affected by the spontaneous respiratory movement of the tested subjects or the pressure set in a positive pressure ventilator. The measurement value obtained by this method is accurate only when the tested subject is in a sedative state.

Another estimating method comprises determining global end-diastolic cardiac volume by a technique of transpulmonary thermodilution, wherein cardiac output is monitored by a peripherally induced continuous cardiac output monitoring apparatus (PiCCO), in which a central venous catheter and a thermister-tipped femoral arterial catheter are used. More specifically, cardiac volume is estimated by injecting a predetermined amount of iced saline through the central venous catheter and measuring the temperature change of the blood in a femoral artery. If the cardiac output is larger, the influence on the blood temperature by the fixed amount of iced water will be smaller and the change in the blood temperature will be less. However, this method needs infusion of about 10 to 15 ml of iced saline into the right heart of the tested subject per each measurement, which is inconvenient for the patients who need long-term hemodynamic monitoring. In addition, cardiac volume does not always correspond to stroke volume. For the subjects with good myocardial contractility, their stroke volume can reach normal value even if they have smaller cardiac volume. In the contrast, for the subjects with compromised myocardial contractility, their stroke volume may not reach normal value even if they have larger cardiac volume.

Further another method for estimating cardiac function utilizes the pulse pressure (difference between systolic pressure and diastolic pressure) measured by an arterial catheter to assess cardiac blood volume. In this method, cardiac function is estimated by measuring the variation in beat-by-beat stroke volume caused by intrathoracic pressure change in the process of respiration (heart-lung interaction). In case that cardiac output reduces due to decreased cardiac blood volume, heart-lung interaction will exert more influence on cardiac stroke volume, which is reflected by a higher pulse pressure variation. Therefore, pulse pressure variation can be used in estimating cardiac blood volume and cardiac function. Conventionally, pulse pressure is obtained by recording the maximal value (max) and the minimal value (min) of arterial blood pressure during a monitoring period of 7.5 seconds, calculating the mean value of the maximal and minimal values, then calculating the pulse pressure variation according to the equation of (max-min)/mean. The larger pulse pressure variation represents smaller cardiac blood volume, and the smaller pulse pressure variation represents larger cardiac blood volume. However, this method is only applicable in patients in a state of general anesthesia or sedation because the pulse pressure variation obtained by this method mainly depends on the extreme values, which are greatly affected by, for example, cough or spontaneous breathing movements of the tested subject. If the subject is not in a sedative state, the accuracy of this method in estimating heart-lung interaction will be greatly reduced.

Other estimating methods include, for example, endocardiography and nuclear scanning. Endocardiography is used to measure the volume of cardiac chambers. It should be conducted by a doctor or a technician skilled in the art and is time- and labor-consuming; therefore, it is not suitable for use in the cases requiring consecutively monitoring cardiac function. Nuclear scanning requires using radioactive isotopes and hence is not applicable in some patients.

The currently used methods for estimating cardiac blood volume usually require the patients to be in a state of general anesthesia or sedation; however, it is highly hazardous for a shock patient to receive general anesthesia and sedation. In addition, the current methods are not satisfactory in the terms of accuracy, safety, radiation exposure level and feasibility. Therefore, it is highly desired to develop a dynamic, accurate method for estimating cardiac function, which is applicable in all patients, including those who are not in a state of general anesthesia and sedation, and is suitable for use in consecutively monitoring cardiac function.

### SUMMARY OF THE INVENTION

One main object of the present invention is to provide a spectrum analytical method for quantifying heart-lung interaction, wherein a heart-associated monitoring signal is used to estimate cardiac function. In view that the events which incidentally affect heart-lung interaction, for example, cough or spontaneous respiratory movement, occur at a low frequency, estimation of cardiac function can be performed in patients who are not in a state of general anesthesia or sedation by choosing spectrum signals within a specified frequency band according to the present invention.

Another object of the present invention is to use the result of quantification of heart-lung interaction as a predictor of cardiac function. During inspiratory period, lung expands and compresses heart; as a result, stroke volume increases because intrathoracic positive pressure is exerted on left ventricle. However, the blood volume injected into pulmonary arteries will be reduced under such positive pressure, which, in turn, may affect the stroke volume of left ventricle on next heart beat. Therefore, inspiratory and expiratory movements may change intrathoracic pressure, which may affect contraction of heart; in turn, may cause variation in beat-by-beat left ventricular stroke volume. The variation value may vary with the myocardial function of the tested subjects; therefore, the result of quantification of heart-lung interaction can be used as a predictor of cardiac function.

To achieve the above object, the present invention provides a spectrum analytical method for quantifying heart-lung interaction, comprising performing spectrum analysis of arterial blood pressure signals within a time domain by the following steps:

(a) transforming the arterial blood pressure signals to pulse pressure signals;
(b) subjecting the pulse pressure signals to spectrum transform to obtain power spectrum signals;
(c) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.

In the above method, there are at least 10 respiratory cycles in the time domain. The pulse pressure signals are obtained by the following equation:

PPₙₒᵣₘ=(PP−PPₘₑₐₙ)/PPₘₑₐₙ

wherein PPₙₒᵣₘ is a normalized pulse pressure signal, PP is an arterial blood pressure signal, and PPₘₑₐₙ is a mean value of the arterial blood pressure signals within the time domain. The integrated energy value of the power spectral densities equal to or less than 4.62×10⁻⁴ (min⁻¹) indicates that the cardiac blood volume is sufficient.

To achieve the above object, the present invention provides a spectrum analytical method for quantifying heart-lung interaction, comprising performing spectrum analysis of stroke volume signals within a time domain by the following steps:

(a) subjecting the stroke volume signals to spectrum transform to obtain power spectrum signals;
(b) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.
In the above method, there are at least 10 respiratory cycles in the time domain. The integrated energy value of the power spectral densities equal to or less than 4.62×10⁻⁴ (min⁻¹) indicates that the cardiac blood volume is sufficient.

To achieve the above object, the present invention provides a spectrum analytical method for quantifying heart-lung interaction, comprising performing spectrum analysis of blood flow signals within a time domain by the following steps:

(a) transforming the blood flow signals to blood flow difference signals;
(b) subjecting the blood flow difference signals to spectrum transform to obtain power spectrum signals;
(c) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.

In the above method, there are at least 10 respiratory cycles in the time domain. The blood flow difference signals are obtained by the following equation:

BFₙₒᵣₘ=(BF−BFₘₑₐₙ)/BFₘₑₐₙ

wherein BFₙₒᵣₘ is a normalized blood flow difference signal, BF is a blood flow signal, and BFₘₑₐₙ is a mean value of blood flow signals within the time domain. The integrated energy value of the power spectral densities equal to or less than 4.62×10⁻⁴ (min⁻¹) indicates that the cardiac blood volume is sufficient.

To achieve the above object, the present invention provides a spectrum analytical method for quantifying heart-lung interaction, comprising performing spectrum analysis of blood flow velocity signals within a time domain by the following steps:

(d) transforming the blood flow velocity signals to blood flow velocity difference signals;
(e) subjecting the blood flow velocity difference signals to spectrum transform to obtain power spectrum signals;
(f) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.

In the above method, there are at least 10 respiratory cycles in the time domain. The blood flow velocity difference signal is obtained by the following equation:

BFVₙₒᵣₘ=(BFV−BFVₘₑₐₙ)/BFVₘₑₐₙ

wherein
BFVₙₒᵣₘ is a normalized blood flow velocity difference signal,
BFV is a blood flow velocity signal,
BFVₘₑₐₙ is a mean value of blood flow velocity signals within the time domain.
The integrated energy value of the power spectral densities equal to or less than 4.62×10⁻⁴ (min⁻¹) indicates that the cardiac blood volume is sufficient.

### BRIEF DESCRIPTION OF DRAWINGS

FIG. 1 is a flow chart of one embodiment of the present invention.

FIG. 2 shows a curve of arterial blood pressure signals vs time in the first embodiment of the present invention.

FIGS. 3A˜3C show power spectral density distribution curves transformed from pulse pressure signals under different conditions in the first embodiment.

FIG. 4 shows curves of arterial blood pressure signals and stroke volume signals respectively versus time in the first embodiment.

FIG. 5 shows a curve of power spectral density for stroke volume vs power spectral density for pulse pressure.

FIG. 6 shows a curve of blood flow signals vs time in the second embodiment.

FIGS. 7A˜7C show power spectral density distribution curves transformed from blood flow difference signals under different conditions in the second embodiment.

FIG. 8 shows a curve of the square root of the energy of power spectral density for blood flow vs. blood loss in the second embodiment in which 6 piglets were subjected to an exsanguination experiment.

FIG. 9 shows a curve of blood flow velocity signals vs time in the third embodiment.

FIGS. 10A˜10C show power spectral density distribution curves transformed from blood flow velocity difference signals under different conditions in the third embodiment.

FIG. 11 shows a curve of the square root of the energy of power spectral density for blood flow velocity vs change percentage in cardiac output in the third embodiment in which 9 piglets were subjected to an exsanguination experiment.

### DETAILED DESCRIPTION OF INVENTION

To understand the objects, features and effects of the present invention, the invention is illustrated by the following examples in reference to the appended drawings.

The spectrum analytical method for quantifying heart-lung interaction according to the present invention can utilize various cardiac output-related signals, such as arterial blood pressure (ABP), stroke volume (SV), blood flow (BF), blood flow velocity (BFV) etc. Any devices for monitoring these signals can be used in the spectrum analytical method according to the present invention.

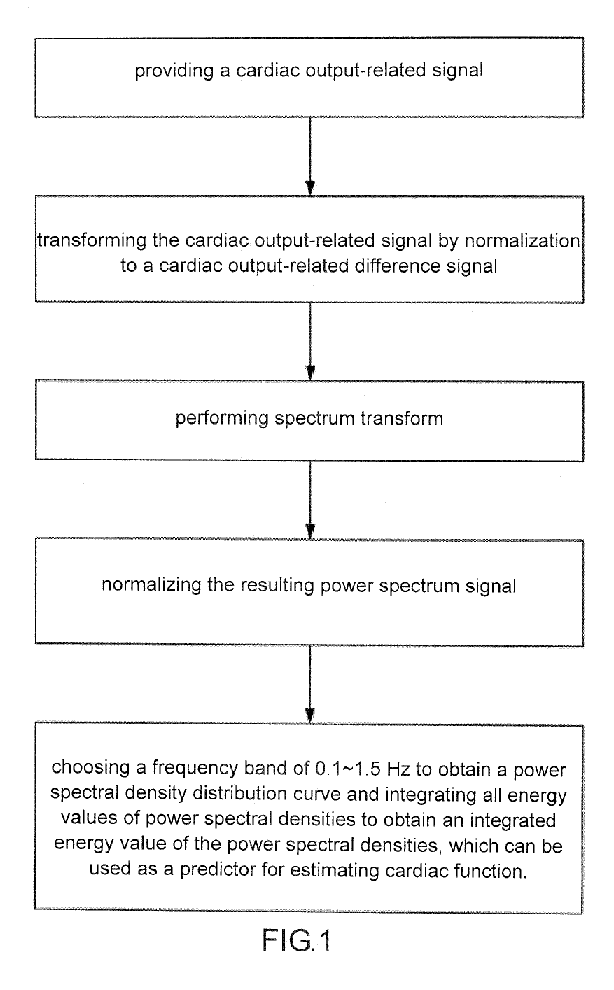

Reference is made to FIG. 1, which is a flow chart of one embodiment of the present invention. First, a cardiac output-related signal is provided; transforming the cardiac output-related signal by normalization to a cardiac output-related difference signal; performing spectrum transform; normalizing the resulting power spectrum signal; finally, choosing a frequency band of 0.1˜1.5 Hz to obtain a power spectral density distribution curve and integrating the energy values of all power spectral density to obtain an integrated all energy values of the power spectral densities, which can be used as a predictor for estimating cardiac function.

Spectrum transform can be performed in any mode suitable for use in transforming a time domain signal to a frequency domain signal. These modes include, but are not limited to, discrete Fourier transform (DFT), fast Fourier transform (FFT), discrete cosine transform (DCT), discrete Hartley transform (DHT) and discrete wavelet transform (DWT). In the embodiments of the present invention, the power spectral density (PSD) distribution curve is obtained by fast Fourier transform using a Matlab computing program according to Welch's method. The functional equation used in the Matlab computing program is as follows:

[Pxx,f]=pwelch(xn,nfft,fs,window,noverlap)

wherein “xn” represents a signal sequence, “nft” represents the length of fast Fourier transform (FFT); “fs” represents the sampled frequency domain; “window” represents the chosen window function, which must be smaller than or equal to “nfft”; “noverlap” represents the overlapped length of each segment in estimation of power spectral density, which must be smaller than “nft”; “Pxx” represents the computed power spectral density, “f” represents frequency coordinate, “Pxx” and “f” are respectively the longitudinal coordinate and the horizontal coordinate in the power spectral density distribution curve.

Before obtaining the power spectral density (PSD) distribution curve by performing fast Fourier transform using a Matlab computing program, the input signals should be processed to unify the time intervals between two sequential signals such that Fourier transform can be smoothly performed. In all of the embodiments of the present invention, the time intervals between two sequential signals, for pulse pressure signals, blood flow difference signals and blood flow velocity difference signals, are unified by, for example, tertiary curve interpolation method. Other methods for unifying the time intervals, including nearest neighbor interpolation, linear interpolation, cloud interpolation etc., are also suitable for use in the present invention. The application of the above interpolation method in the functional equation of Matlab computing program is well known in the art, therefore, the detailed description thereof is omitted.

After obtaining the power spectrum signals, a frequency band of 0.1˜1.5 Hz is chosen and analyzed to estimate cardiac function. For an adult, preferably, a frequency band of 0.15˜0.75 Hz, which corresponds to respiratory rate of 9˜45 breaths per minute, is chosen.

In the first embodiment of the present invention, the spectrum analytical method for quantifying heart-lung interaction is used in monitoring of arterial blood pressure. The arterial blood pressure is measured by a conventional device for monitoring cardiopulmonary volume or any other devices for monitoring arterial blood pressure.

First, arterial blood pressure signals in a time domain are obtained by using a device for monitoring arterial blood pressure, then the arterial blood pressure signals are transformed to pulse pressure (PP) signals by normalization according to the following equation (1):

PPₙₒᵣₘ=(PP−PPₘₑₐₙ)/PPₘₑₐₙ (1)

wherein
PPₙₒᵣₘ is a normalized pulse pressure signal,
PP is an arterial blood pressure signal,
PPₘₑₐₙ is a mean value of arterial blood pressure signals within the time domain.
In order to obtain sufficient arterial blood pressure signals for analysis, there are 2 or more, preferably 10 or more, respiratory cycles in the time domain. The measuring time is about 1 minute or more.

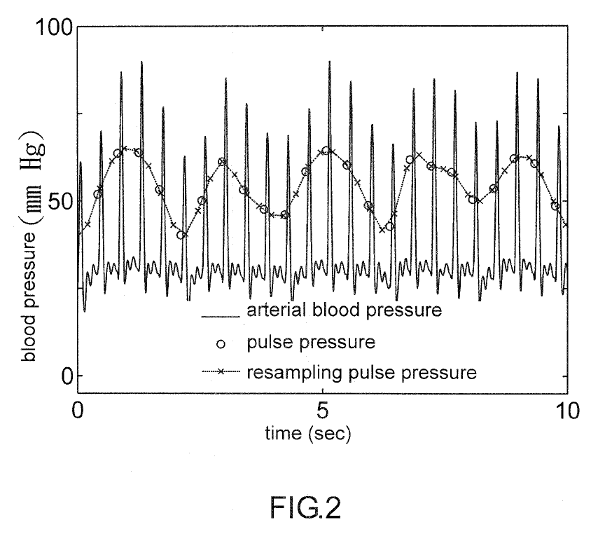

Reference is made to FIG. 2. FIG. 2 shows a curve of arterial blood pressure signal vs. time in the first embodiment, wherein the solid line represents arterial blood pressure signals, the dotted line represents resampling pulse pressure signals obtained by interpolation method. It can be seen from FIG. 2 that the arterial blood pressure signals go up and down in a regular form. This is due to change in intrathoracic pressure, which, in turn, leads to change in systolic pressure and cardiac output.

Next, the pulse pressure signal are subjected to spectrum transform by fast Fourier transform to obtain power spectrum signals, which are normalized with respective to energy. In one embodiment, the power spectral energy density is normalized with respective to the sampled time domain. Finally, all energy values of all power spectral densities over the chosen frequency band are integrated to obtain an integrated energy value of power spectral densities.

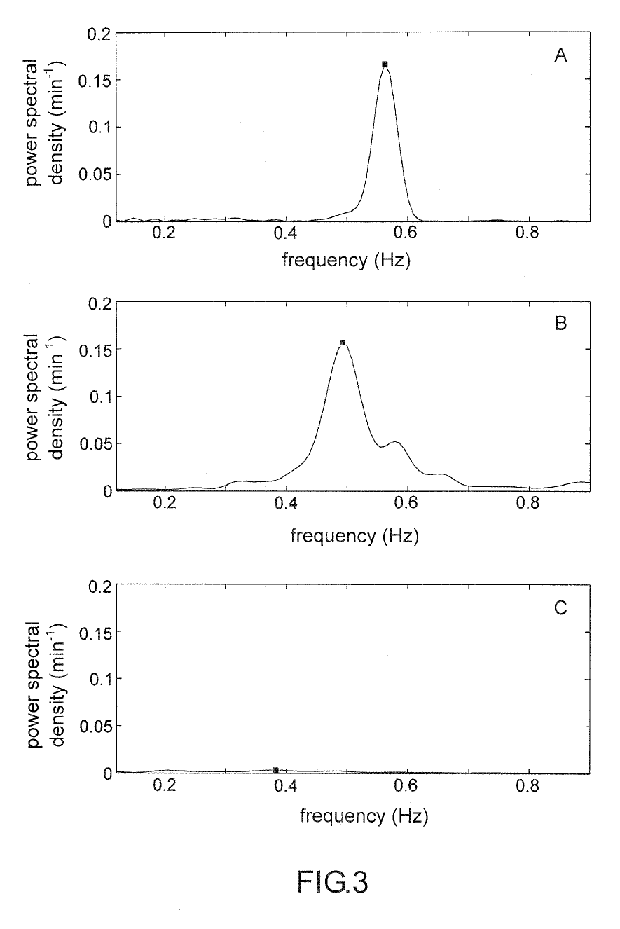

Reference is made to FIGS. 3A to 3C. FIGS. 3A to 3C show power spectral density distribution curves transformed from pulse pressure signals under different conditions in the first embodiment. FIG. 3A represents a power spectral density distribution curve transformed from pulse pressure signals in an anesthetized subject who received positive pressure ventilation by a ventilator. FIG. 3B represents a power spectral density distribution curve transformed from pulse pressure signals in a non-anesthetized, mechanically ventilated subject who are allowed to trigger supported breath freely. FIG. 3C represents a power spectral density distribution curve transformed from pulse pressure signals in a subject with higher blood volume.

As shown in FIG. 3B, a distinct peak appears even in the case that the tested subject is not in a state of general anesthesia or sedation. Furthermore, FIGS. 3A and 3B correspond to the cases that the intrathoracic pressure of the tested subjects significantly affects the cardiac output; while FIG. 3C, in which no distinct peak appears, correspond to the case that the intrathoracic pressure of the tested subjects does not significantly affects the cardiac output.

The power spectrum is used to analyze the result of quantification of the heart-lung interaction, which can estimate the cardiac blood volume of the tested subject by a power spectral density distribution curve. According to the spectrum analytical method of the present invention, a frequency band with the suitable frequency range, for example, 0.15˜0.75 Hz as shown in FIGS. 3A˜3C, is chosen, such that the signals from incidental events such as cough or abrupt alteration of arterial blood pressure, which usually occurs at a low frequency, does not fall in the chosen frequency band. When breath frequency increases, the peak will shift toward higher frequency in the FIGS. 3A˜3C.

Under the same intrathoracic pressure, hearts with smaller blood volume will be more significantly affected by intrathoracic pressure. Therefore, in case that intrathoracic pressure goes up and down with respiratory movement, larger pulse pressure variability usually represents smaller cardiac blood volume, i.e. smaller preload. In the contrast, in the case that the heart of the tested subject has sufficient blood volume, the influence of heart-lung interaction on the heart will decrease, as a result, pulse pressure variability will become smaller. Furthermore, the energy value of the power spectral density will vary with pulse pressure variability. As pulse pressure variability increases, the integrated energy value of power spectral densities becomes higher. Through choosing a frequency band of 0.1˜1.5 Hz, preferably 0.15˜0.75 Hz for an adult, a power spectral density distribution curve from arterial blood pressure signals is obtained. All energy values of power spectral densities over the chosen frequency band are integrated. The integrated energy value of the power spectral densities smaller than or equal to 4.62×10⁻⁴ (min.⁻¹) means that the integrated value of the variation in pulse pressure is small. This reveals that the influence of intrathoracic pressure on the cardiac output of the tested subject is small and the tested subject has sufficient cardiac blood volume since the pulse pressure is not significantly affected by respiratory movement or heart-lung interaction.

In the first embodiment of the present invention, the arterial blood pressure signals are also transformed to stroke volume signals, which can be analyzed by the spectrum analytical method of the present invention. The transform is performed according to the following equation (2):

$$SV = cal \cdot [ \frac{SA}{SVR} + {\int }_{systole}^{ } ( C ( p ) \cdot \frac{d P}{d t} ) d t ] \qquad ( 2 )$$

wherein SV is stroke volume, SA is the integrated area under arterial blood pressure waveform, SVR is systemic vascular resistance, C(p) is compliance of vascular system (mainly arota), dP/dt is derivative of systolic arterial blood pressure waveform over time, and cal is a calibrated value for respective tested subject. By the above equation (2), a curve of arterial blood pressure vs time can be transformed to a curve of beat-by-beat stroke volume vs time.

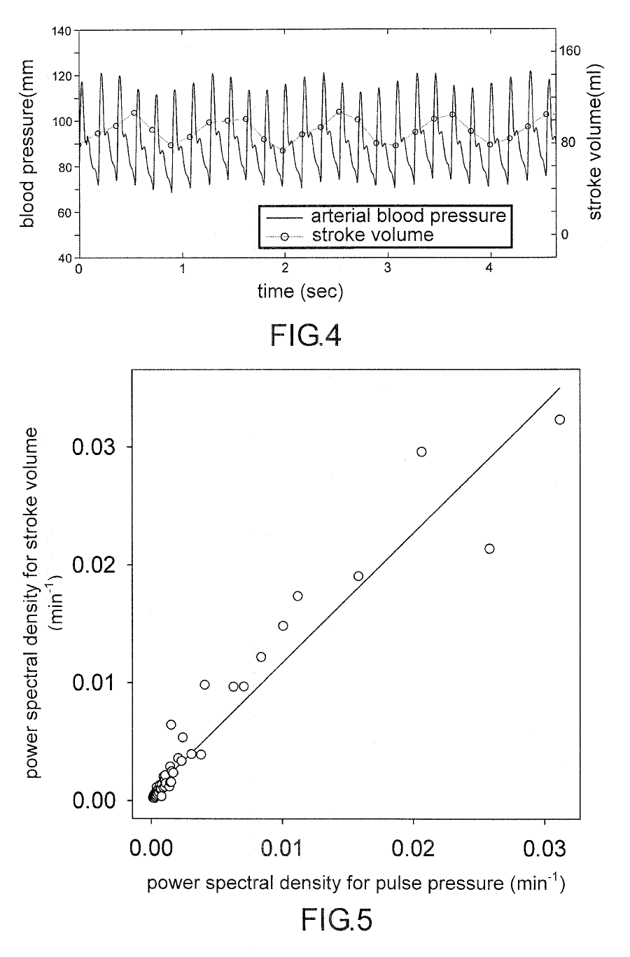

Reference is made to FIG. 4. FIG. 4 shows curves of arterial blood pressure and stroke volume respectively versus time, wherein the solid line represents arterial blood pressure signals, the dotted line represents the stroke volume signals obtained by interpolation method after transforming the arterial blood pressure signals. The spectrum analytical results of the both two signals are as shown in FIG. 5.

Reference is made to FIG. 5. FIG. 5 shows a curve of the power spectral density for stroke volume vs the power spectral density for pulse pressure, wherein the spectrum analytical results of the pulse pressure and the stroke volume, which are expressed as the mean values of the data taken from 9 pigs, are compared. The correlation coefficient (r-square) between these two parameters is 0.95, which indicates that there is a linear relationship between the pulse pressure variability and the stroke volume variability. In other words, these two parameters can be used “interchangeably”.

In the second embodiment of the present invention, the spectrum analytical method for quantifying heart-lung interaction is based on monitoring of blood flow. The blood flow is measured by a conventional infrared plethysmography monitoring device or any other device suitable for monitoring blood flow. The infrared plethysmography monitoring device monitors blood flow by measuring the absorption of infrared light by hemoglobin.

As stated before, first, blood flow signals in a time domain are taken, then transformed to blood flow difference signals by normalization according to the following equation (3):

BFₙₒᵣₘ=(BF−BFₘₑₐₙ)/BFₘₑₐₙ (3)

wherein
BFₙₒᵣₘ is a normalized blood flow difference signal,
BF is a blood flow signal,
BFₘₑₐₙ is a mean value of the blood flow signals within the time domain.
In order to obtain sufficient blood flow signals for analysis, there are 2 or more, preferably 10 or more, respiratory cycles in the time domain. The measuring time is about 1 minute or more.

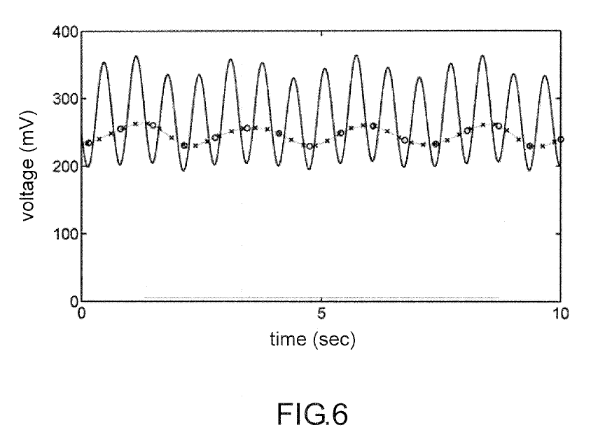

Reference is made to FIG. 6. FIG. 6 shows a curve of blood flow vs. time. In the infrared monitoring device, absorption of infrared light by hemoglobin is transformed to voltage signals, which is shown at Y-axis in FIG. 6. The solid line represents blood flow signals and the dotted line represents the blood flow difference signals obtained by interpolation method. It can be seen from FIG. 6 that the blood flow signals go up and down in a regular form. This is due to change in the intrathoracic pressure, which, in turn, leads to change in systolic pressure and cardiac output.

Similarly, the blood flow difference signals are subjected to spectrum transform by fast Fourier transform to obtain power spectrum signals and the power spectrum is normalized with respective to energy. In one embodiment, the power spectral density is normalized with respective to the sampled time domain, then a frequency band of 0.1˜1.5 Hz, preferably 0.15˜0.75 Hz for an adult is chosen to obtain a power spectral density distribution curve for the blood flow difference signals. Finally, all energy values of power spectral densities over the chosen frequency band are integrated to obtain an integrated energy value of power spectral densities.

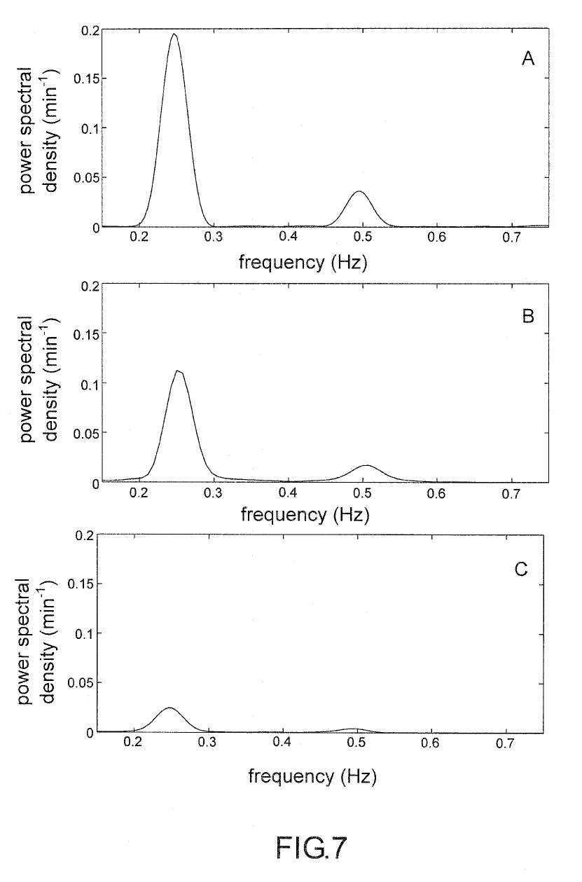

Reference is made to FIGS. 7A to 7C. FIGS. 7A to 7C show power spectral density distribution curves transformed from blood flow difference signals under different conditions in the second embodiment. FIGS. 7A to 7C represent power spectral density distribution curves transformed from blood flow difference signals in an anesthetized subjects who received positive pressure mechanical ventilation As stated above, the integrated energy value of the power spectral densities smaller than or equal to 4.62×10⁻⁴ (min.⁻¹) means that the integrated value of the variation in blood flow difference is small. This reveals that the influence of intrathoracic pressure on the cardiac output of the tested subject is small and the tested subject has sufficient cardiac blood volume since the blood flow is not significantly affected by respiratory movement or heart-lung interaction. In FIG. 7, the smaller waveform (peak) appearing at a frequency as twice as main frequency is due to the harmonic wave produced by the spectral analysis.

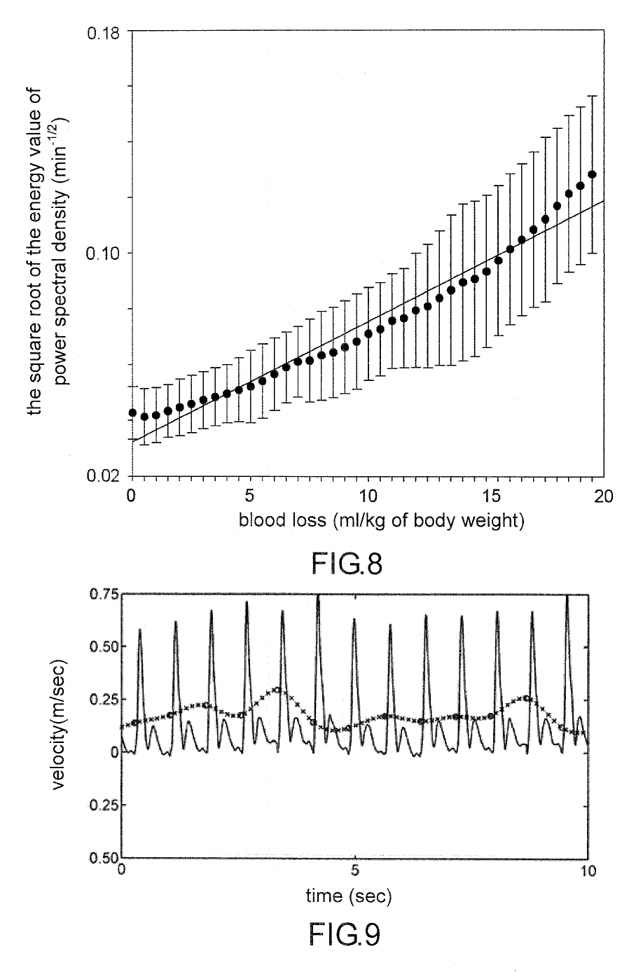

Reference is made to FIG. 8. FIG. 8 shows a curve of the square root of the energy of blood flow power spectral density vs. blood loss in the second embodiment in which 6 piglets are subjected to a exsanguination experiment. The square root of the energy is used to manifest the relationship between the blood flow variability and the blood loss. The six piglets were exsanguinated under general intravenous anesthesia and artificial ventilation. The frequency band of 0.1550.75 Hz was chosen from the power spectrum. It can be seen from FIG. 8 that there is a linear relationship between the square root of the energy of power spectral density and blood loss. Blood loss can be used to estimate the cardiac blood volume (preload). The more the blood loss is (namely the smaller preload is), the smaller the cardiac stroke volume is and the larger the square root of the energy of power spectral density is. Therefore, change in the square root of the energy of power spectral density can reflect the change in cardiac function during exsanguination process, and hence can be used to indicate the degree of blood loss.

In the third embodiment of the present invention, the spectrum analytical method for quantifying heart-lung interaction is based on monitoring of blood flow velocity. The blood flow velocity can be obtained by measuring the potential difference between the two electrodes when blood flows through these two electrodes, or measured by a Doppler ultrasound device or any other device suitable for monitoring blood flow velocity. Through the above method, blood flow velocity can be non-invasively measured without contacting blood. The Doppler ultrasound device is used to monitoring the blood flow velocity in femoral arteries.

As stated above, blood flow velocity signals in a time domain are taken and then transformed to blood flow velocity difference signals by normalization according to the following equation (4):

BFVₙₒᵣₘ=(BFV−BFVₘₑₐₙ)/BFVₘₑₐₙ

wherein
BFVₙₒᵣₘ is a normalized blood flow velocity difference signal,
BFV is a blood flow velocity signal,
BFVₘₑₐₙ is a mean value of blood flow velocity signals within the time domain.
In order to obtain sufficient blood flow velocity signals for analysis, there are 2 or more, preferably 10 or more, respiratory cycles in the time domain. The measuring time is about 1 minute or more.

Reference is made to FIG. 9. FIG. 9 shows a curve of blood flow velocity vs. time in the third embodiment. In the Figure, the solid line represents blood flow velocity signals and the dotted line represents the blood flow velocity difference signals obtained by interpolation method. It can be seen from FIG. 9 that the blood flow velocity signals go up and down in a regular form. This is due to change in the intrathoracic pressure, which, in turn, leads to change in systolic pressure and cardiac output.

Similarly, the blood flow velocity difference signals are subjected to spectrum transform by fast Fourier transform to obtain power spectrum signals and the power spectrum is normalized with respective to energy. In one embodiment, power spectral density is normalized with respective to the sampled time domain, then a frequency band of 0.1˜1.5 Hz, preferably 0.15˜0.75 Hz for an adult is chosen to obtain a power spectral density distribution curve for the blood flow difference signals. Finally, all energy values of power spectral densities over the chosen frequency band are integrated to obtain an integrated energy value of power spectral densities.

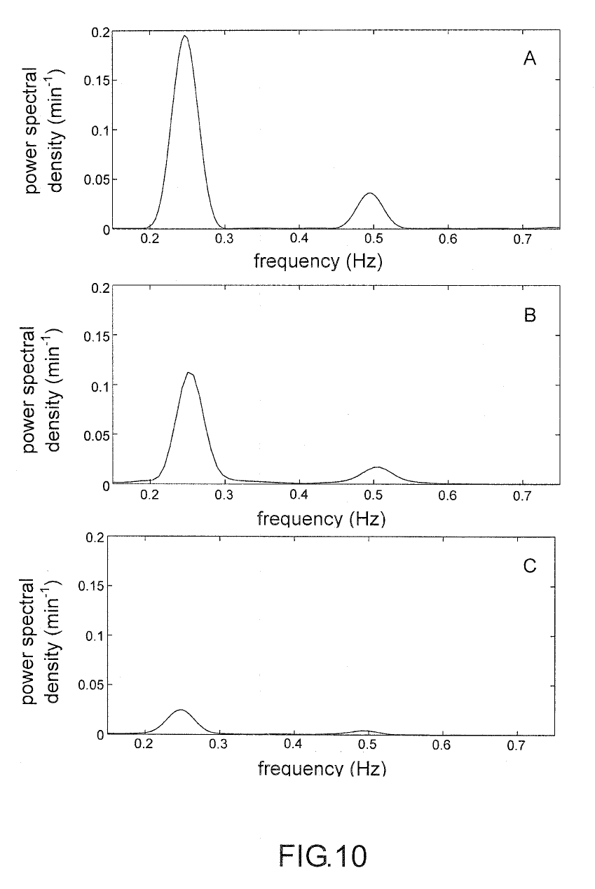

Next, reference is made to FIGS. 10A to 10C. FIGS. 10A to 10C show power spectral density distribution curves transformed from the blood flow velocity difference signals under different conditions in the third embodiment. FIG. 10A to 10C represent power spectral density distribution curves transformed from the blood flow velocity difference signals in anesthetized subjects who received positive pressure ventilation by a ventilator. As stated above, the integrated energy value of the power spectral densities smaller than or equal to 4.62×10⁻⁴ (min.⁻¹) means that the integrated value of the variation in blood flow velocity difference is small. This reveals that the influence of intrathoracic pressure on the cardiac output of the tested subject is small and the tested subject has sufficient cardiac blood volume since the blood flow velocity is not significantly affected by respiratory movement or heart-lung interaction.

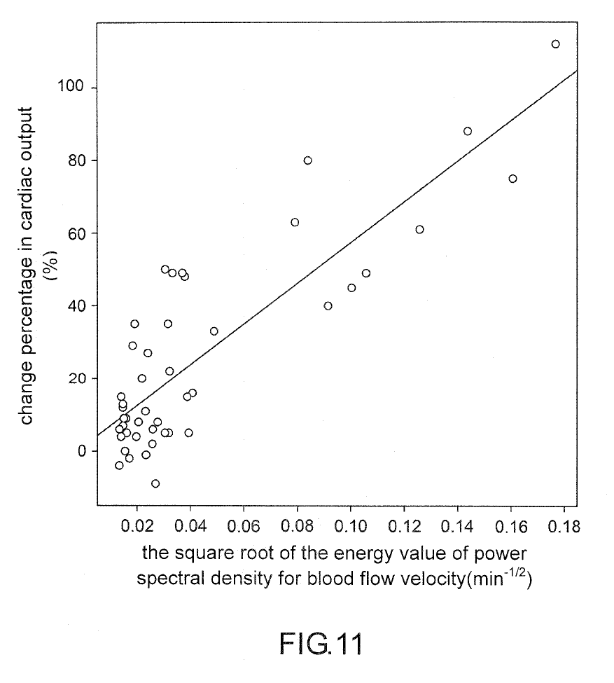

Reference is made to FIG. 11. FIG. 11 shows a curve of the square root of the energy value of power spectral density for blood flow velocity vs. change percentage of cardiac output in the third embodiment in which 9 piglets are subjected to a exsanguination experiment. In this experiment, the nine piglets, after exsanguinated under general intravenous anesthesia and artificial ventilation, were treated with 10% hydroxyethyl starch for intravascular volume expansion. Namely, 8 ml/kg of body weight of 10% hydroxyethyl starch was administered to the piglets at each stage. It can be seen from FIG. 11 that, within the frequency band of 0.15-75 Hz, there is a linear relationship between the square root of the energy value of power spectral density and the change percentage in cardiac output after the plasma substitute is administered. From FIGS. 2 to 9, it is can be seen that the spectrum analytical method of the present invention is applicable in the subjects who are or are not in a state of general anesthesia.

In conclusion, the cardiac output-related parameters can be used as a predictor of cardiac function and are helpful in diagnosis. The spectrum analytic method of the present invention can be used in analyzing the measurements obtained from any devices for monitoring these cardiac output-related parameters. Through performing spectrum transform and choosing a suitable frequency band from the spectrum, the spectrum analysis according to the present invention can be conducted without need of anesthetizing or sedating the tested subjects; furthermore, the spectrum analytical result of quantification of heart-lung interaction can be used as a predictor of the cardiac function of the tested subjects.

The present invention has been disclosed by the above preferred embodiments. It can be understood by the person skilled in the art that these embodiments are merely for illustration of the present invention, but are not considered as a limitation thereto. It should be noted that all the equivalent alterations or replacements of these embodiments fall in the scope of the present invention. The scope of the present invention is defined by the appended claims.

What is claimed is:

1. A spectrum analytical method for quantifying heart-lung interaction, comprising performing spectrum analysis of arterial blood pressure signals within a time domain by the following steps:
(a) transforming the arterial blood pressure signals to pulse pressure signals;
(b) subjecting the pulse pressure signals to spectrum transform to obtain power spectrum signals;
(c) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.

2. The spectrum analytical method of claim 1, wherein there are at least 10 respiratory cycles in the time domain.

3. The spectrum analytical method of claim 1, wherein the pulse pressure signal in the step (a) is obtained according to the following equation:
PPₙₒᵣₘ=(PP−PPₘₑₐₙ)/PPₘₑₐₙ
wherein
PPₙₒᵣₘ is a normalized pulse pressure signal,
PP is an arterial blood pressure signal,
PPₘₑₐₙ is a mean value of the arterial blood pressure signals within the time domain.

4. The spectrum analytical method of claim 1, wherein the pulse pressure signals are subjected to spectrum transform by fast Fourier transform in the step (b).

5. The spectrum analytical method of claim 1, wherein the power spectrum signals in the step (b) are normalized.

6. The spectrum analytical method of claim 1, wherein the integrated energy value of the power spectral densities in the step (c) equal to or less than 4.62×10⁻⁴ (min⁻¹) indicates that the cardiac blood volume is sufficient.

7. A spectrum analytical method for quantifying heart-lung interaction, comprising performing spectrum analysis of stroke volume signals within a time domain by the following steps:
(a) subjecting the stroke volume signals to spectrum transform to obtain power spectrum signals;
(b) choosing a frequency band of 0.1˜1.5 Hz from the power spectrum signals to obtain a power spectral density distribution curve, integrating all energy values of the power spectral densities over the chosen frequency band to obtain an integrated energy value of the power spectral densities, which is used as a predictor of cardiac function.

8. The spectrum analytical method of claim 7, wherein there are at least 10 respiratory cycles in the time domain.

9. The spectrum analytical method of claim 7, wherein the stroke volume signals are subjected to spectrum transform by fast Fourier transform in the step (b).

10. The spectrum analytical method of claim 7, wherein the stroke volume signal in the step (b) is normalized.

11. The spectrum analytical method of claim 7, wherein the integrated energy value of the power spectral densities in the step (c) equal to or less than 4.62×10⁻⁴ (min⁻¹) indicates that the cardiac blood volume is sufficient.

12. A spectrum analytical method for quantifying heart-lung interaction, comprising performing spectrum analysis of a blood flow signals within a time domain by the following steps:
(a) transforming the blood flow signals to a blood flow difference signals;
(b) subjecting the blood flow difference signals to spectrum transform to obtain power spectrum signals;
(c) choosing a frequency band of 0.1˜1.5 Hz from the power spectrum signals to obtain a power spectral density distribution curve over the chosen frequency band, integrating all energy values of the power spectral densities over the chosen frequency band to obtain an integrated energy value of the power spectral densities, which is used as a predictor of cardiac function.

13. The spectrum analytical method of claim 12, wherein there are at least 10 respiratory cycles in the time domain.

14. The spectrum analytical method of claim 12, wherein the blood flow difference signals in the step (a) are obtained by the following equation:
BFₙₒᵣₘ=(BF−BFₘₑₐₙ)/BFₘₑₐₙ
wherein
BFₙₒᵣₘ is a normalized blood flow difference signal,
BF is a blood flow signal,
BFₘₑₐₙ is a mean value of the blood flow signals within the time domain.

15. The spectrum analytical method of claim 12, wherein the blood flow difference signals are subjected to spectrum transform by fast Fourier transform in the step (b).

16. The spectrum analytical method of claim 12, wherein the power spectrum signal in the step (b) is normalized.

17. The spectrum analytical method of claim 12, wherein the integrated energy value of the power spectral densities in the step (c) equal to or less than 4.62×10⁻⁴ (min⁻¹) indicates that the cardiac blood volume is sufficient.

18. A spectrum analytical method for quantifying heart-lung interaction, comprising performing spectrum analysis of a blood flow velocity signals within a time domain by the following steps:
(a) transforming the blood flow velocity signals to blood flow velocity difference signals;
(b) subjecting the blood flow velocity difference signals to spectrum transform to obtain power spectrum signals;
(c) choosing a frequency band of 0.1˜1.5 Hz from the power spectrum signals to obtain a power spectral density distribution, integrating all energy values of the power spectral densities over the chosen frequency band to obtain an integrated energy value of the power spectral densities, which is used as a predictor of cardiac function.

19. The spectrum analytical method of claim 18, wherein there are at least 10 respiratory cycles in the time domain.

20. The spectrum analytical method of claim 18, wherein the blood flow velocity difference signals in the step (a) are obtained by the following equation:
BFVₙₒᵣₘ=(BFV−BFVₘₑₐₙ)/BFVₘₑₐₙ
wherein
BFVₙₒᵣₘ is a normalized blood flow velocity difference signal,
BFV is a blood flow velocity signal,
BFVₘₑₐₙ is a mean value of blood flow velocity signals within the time domain.

21. The spectrum analytical method of claim 18, wherein the blood flow velocity difference signals are subjected to spectrum transform by fast Fourier transform in the step (b).

22. The spectrum analytical method of claim 18, wherein the power spectrum signals in the step (b) are normalized.

23. The spectrum analytical method of claim 18, wherein the integrated energy value of the power spectral densities in the step (c) equal to or less than 4.62×10⁻⁴ (min⁻¹) indicates that the cardiac blood volume is sufficient.
